(19) United States
(12) Patent Application Publication
(10) Pub. No.: US 2021/0154117 A1
(43) Pub. Date: May 27, 2021

(54) ESTERAMINE SALTS

(71) Applicant: BASF SE, Ludwigshafen am Rhein (DE)

(72) Inventors: Sophia EBERT, Ludwigshafen am Rhein (DE); Bjoern LUDOLPH, Ludwigshafen am Rhein (DE); Dawid MARCZEWSKI, Ludwigshafen am Rhein (DE)

(21) Appl. No.: 17/256,821

(22) PCT Filed: Jun. 21, 2019
(86) PCT No.: PCT/EP2019/066537

§ 371 Date: Dec. 29, 2020

(30) Foreign Application Priority Data
Jun. 29, 2018 (EP) 18180689.4

(51) Int. Cl. A61K 8/46; A61K 8/41; A61Q 5/02; A61Q 19/10; C07C 303/32; C07C 309/31; C07C 219/08; C07C 219/06; C08G 59/50; C11D 11/00; C11D 3/34

(52) U.S. Cl. CPC A61K 8/466; A61K 8/41; A61Q 5/02; A61Q 19/10; C07C 303/32; C11D 3/349; C07C 219/08; C07C 219/06; C08G 59/504; C11D 11/0017; C07C 309/31

(57) ABSTRACT

The present invention relates to an esteramine salt according to the general formula (I):

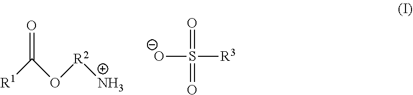

The substituents R¹, R² and R³ are defined below.

The present invention further relates to a process for preparing such an esteramine salt according to general formula (I), wherein a corresponding monocarboxylic acid or an ester thereof are reacted with an aminoalcohol and an at least equimolar amount of a sulfonic acid.

The present invention relates to an esteramine salt according to the general formula (I):

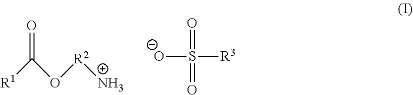

The substituents R¹, R² and R³ are defined below.

The present invention further relates to a process for preparing such an esteramine salt according to general formula (I), wherein a corresponding monocarboxylic acid or an ester thereof are reacted with an aminoalcohol and an at least equimolar amount of a sulfonic acid.

Due to the increasing popularity of easy-care fabrics made of synthetic fibers as well as the increasing energy costs and growing ecological concerns of detergent users, the once popular hot water wash has now taken a back seat to washing fabrics in cold water. Many commercially available laundry detergents are even advertised as being suitable for washing fabrics at 40° C. or 30° C. or even at room temperature. To achieve satisfactory washing result at such low temperatures, i.e. results comparable to those obtained with hot water washes, the demands on low temperature detergents are especially high.

It is known to include certain additives in detergent compositions to enhance the detergent power of conventional surfactants so as to improve the removal of grease stains at temperatures of 60° C. and below.

EP 17180161.6 relates to alkoxylated esteramines and salts thereof. The respective esteramines and salts thereof mandatorily contain fragments based on alkoxy units as well as fragments based on amino acids, such as alanine or glycine. Furthermore, a process for the preparation of such esteramines or salts thereof is disclosed as well as their use in personal care compositions.

U.S. Pat. No. 3,398,163 relates to organic compounds which are useful as non-ionic detergents. The respective organic compounds are ethylene oxide adducts of amino esters. The respective compounds are prepared in a first step by reacting a monocarboxylic acid with a hydroxy-substituted alkyl primary amine in the presence of an acid catalyst. The so obtained intermediate is further reacted in a second step by performing an alkoxylation in order to obtain the organic compounds, which are useful as non-ionic detergents. By consequence, said organic compounds do not contain any primary amine fragments. Furthermore, said organic compounds do not contain any fragments based on organic sulfonic acid anions.

US-A 2010/0298183 relates to an additive for oils that is capable of imprinting oils, such as lubricant base oils with superior wear resistance properties or friction resistance properties, and a lubricant. The specific compounds disclosed therein also comprise esteramines, which may optionally be present as acid addition salts including organic acid salts, such as carboxylates or sulfonates as well as inorganic acid salts, including a hydrochloride or nitrate. However, the specific ester amines disclosed in US-A 2010/0298183 are based on dicarboxylic acids.

J. Geurts et al. (Journal of Applied Polymer Science, Volume 80, 1401-1415 (2001)) relates to the synthesis of new amino-functionalized methacrylates and their use in free radical polymerizations.

DE-A 1 593 962 relates to a process for producing acyloxyalkylamine hydrochlorides from acids and aminoalcohols with gaseous hydrochloric acid. Such compounds are considered as valuable intermediates for the production of further compounds, such as isocyanates by reacting with phosgene. The employed acids are dicarboxylic acids in order to obtain the corresponding hydrochloride salts. Salts based on organic sulfonic acids are not disclosed in DE-A 1 593 962.

Instead of (di)carboxylic acids it is also known to employ esters of carboxylic acids as a starting material in order to obtain esteramines. However, the respective reaction starting with esters of carboxylic acids are usually performed under chemoselective enzymatic synthesis by employing specific enzymes, such as Novozym® 435 (F. Le Joubioux et al.; Journal of Molecular Catalysis B: Enzymatic 95 (2013) 99-110), or by employing fatty acid amide hydrolase (FAAH) as described in Y. Yamano et al. (Bioorganic & Medicinal Chemistry 20 (2012) 3658-3665). Due to the employment of specific enzymes, the respective esteramines are not obtained in form of a salt of an organic sulfonic acid. Furthermore, the respective esteramines are intended to be employed in specific pharmaceutical applications, such as anti-tumor drugs or anti-inflammatory compounds.

There is a continuous need for cleaning compositions that remove grease stains from fabrics and other soiled materials, as grease stains are challenging stains to remove. Conventional cleaning compositions directed to grease removal frequently utilize various amine compounds which tend to show strong negative impacts on whiteness.

As a consequence there is still a continual need for amine compounds which provide grease removal abilities from fabrics and other soiled materials which at the same time do not negatively impact clay cleaning abilities or whiteness. There is a need for compounds having grease cleaning abilities at low temperatures.

The object of the present invention is to provide novel compounds which comply with the above-identified objectives and needs.

The object is achieved by an esteramine salt according to general formula (I)

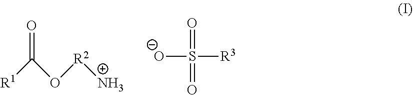

wherein:
R¹ is C₄-C₃₀-alkyl or C₄-C₃₀-alkenyl,
R² is C₃-C₁₂-alkylene or —((CR¹⁰R¹¹)ₒ—CR⁴R⁵—CR⁶R⁷—O)ₘ (CR⁸R⁹)ₙ—,
R³ is C₂-C₃₀-alkyl, C₂-C₃₀-alkenyl or unsubstituted or at least monosubstituted aryl and the substituents are independently selected from C₁-C₃₀-alkyl under the proviso that R³ is not para toluenyl,
R⁴, R⁵, R⁶, R⁷, R⁸, R⁹, R¹⁰ and R¹¹ are independently of each other selected from hydrogen or C₁-C₁₀-alkyl,
m is an integer from 1 to 100,
n is an integer from 2 to 12, and
o is an integer from 0 to 10.

The esteramine salts according to the present invention may be used in cleaning composition, for example in liquid laundry detergents. They lead to improved cleaning performance of said compositions, for example when used in cold water washing conditions. They surprisingly boost grease cleaning performance of liquid laundry detergents, especially under cold water washing conditions. The esteramine salts according to the present invention show improved compatibility in liquid laundry detergent formulations.

For the purposes of the present invention, definitions such as C₁-C₃₀-alkyl, as defined above for, for example, the radical R³ in formula (I), mean that this substituent (radical) is an alkyl radical having from 1 to 30 carbon atoms. The alkyl radical can be either linear or branched or optionally cyclic. Alkyl radicals which have both a cyclic component and a linear component likewise come within this definition. The same applies to other alkyl radicals such as a C₄-C₃₀-alkyl radical or a C₆-C₁₈-alkyl radical. Examples of alkyl radicals are methyl, ethyl, n-propyl, sec-propyl, n-butyl, sec-butyl, isobutyl, 2-ethylhexyl, tert-butyl (tert-Bu/t-Bu), pentyl, hexyl, heptyl, cyclohexyl, octyl, nonyl, decyl or dodecyl.

For the purposes of the present invention, definitions such as C₂-C₃₀-alkenyl, as defined above for, for example, the radical R³ in formula (I), mean that this substituent (radical) is an alkenyl radical having from 2 to 30 carbon atoms. This carbon radical is preferably monounsaturated but can optionally also be doubly unsaturated or multiply unsaturated. As regards linearity, branches and cyclic constituents, what has been said above for C₁-C₃₀-alkyl radicals applies analogously. C₂-C₁₀-alkenyl is, for the purposes of the present invention, preferably vinyl, 1-allyl, 3-allyl, 2-allyl, cis- or trans-2-butenyl, ω-butenyl.

The term “C₃-C₁₂-alkylene” as used herein refers to a saturated, divalent straight chain or branched hydrocarbon chains of 3, 4, 5, 6 or up to 12 carbon groups, examples including propane-1,3-diyl, propane-1,2-diyl, 2-methylpropane-1,2-diyl, 2,2-dimethylpropane-1,3-diyl, butane-1,4-diyl, butane-1,3-diyl (=1-methylpropane-1,3-diyl), butane-1,2-diyl, butane-2,3-diyl, 2-methyl-butan-1,3-diyl, 3-methyl-butan-1,3-diyl (=1,1-dimethylpropane-1,3-diyl), pentane-1,4-diyl, pentane-1,5-diyl, pentane-2,5-diyl, 2-methylpentane-2,5-diyl (=1,1-dimethylbutane-1,3-diyl) and hexane-1,6-diyl.

For the purposes of the present invention, the term “aryl”, as defined above for, for example, the radical R³ in formula (I), means that the substituent (radical) is an aromatic. The aromatic can be a monocyclic, bicyclic or optionally polycyclic aromatic. In the case of polycyclic aromatics, individual rings can optionally be fully or partially saturated. Preferred examples of aryl are phenyl, naphthyl or anthracyl, in particular phenyl.

Within the context of the present invention, those substituents (radicals), such as C₁-C₃₀-alkyl, C₄-C₃₀-alkyl, C₆-C₁₈-alkyl, C₄-C₃₀-alkenyl and/or C₂-C₁₂-alkylene (as well as any other comparable substituent) may be unsubstituted or at least monosubstituted with any further substituent (known to a skilled person), such as alkoxy, amino, hydroxy, carboxy, etc. However, it is preferred within the context of the present invention that said substituents (unless indicated otherwise, for example, for aryl or phenyl) do not contain any further substituents. By consequence, the respective substituent is unsubstituted, which means that it is either straight-chain (linear) or branched. This is in particular the case for the substituents (radicals) R¹, R² and R⁴ to R¹¹. It has to be noted that branched substituents themselves, such as sec-propyl or sec-butyl, are considered within the context of the present invention as being unsubstituted.

The invention is specified in more detail as follows:

The invention relates to an esteramine salt according to general formula (I)

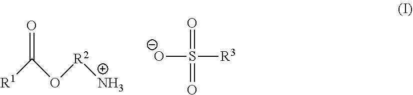

wherein:
R¹ is C₄-C₃₀-alkyl or C₄-C₃₀-alkenyl,
R² is C₃-C₁₂-alkylene or —((CR¹⁰R¹¹)ₒ—CR⁴R⁵—CR⁶R⁷—O)ₘ—(CR⁸R⁹)ₙ—,
R³ is C₂-C₃₀-alkyl, C₂-C₃₀-alkenyl or unsubstituted or at least monosubstituted aryl and the substituents are independently selected from C₁-C₃₀-alkyl under the proviso that R³ is not para toluenyl,
R⁴, R⁵, R⁶, R⁷, R⁸, R⁹, R¹⁰ and R¹¹ are independently of each other selected from hydrogen or C₁-C₁₀-alkyl,
m is an integer from 1 to 100,
n is an integer from 2 to 12, and
o is an integer from 0 to 10.

For the sake of completeness, it is indicated that within general formula (I) individual fragments, which are based on a repetition unit, such as the fragment (CR⁸R⁹)ₙ of the substituent R², may contain an individual substituent, such as R⁸ or R⁹, twice or even more and the definition of such substituents is selected independently from each other. For example, the respective fragment contains for n=3 three carbon atoms and each carbon atom contains one substituent R⁸ and one substituent R⁹.

In such a case, the respective substituents R⁸ and R⁹ may be selected independently from each other for each carbon atom. By consequence, the first carbon atom may contain a substituent R⁸, which is for example H, whereas the second and/or third carbon atom may contain a substituent R⁸, which is for example methyl.

The same principle may apply to any other repetition unit within the compounds according to general formula (I) or within the respective educts to be employed for producing compounds according to formula (I).

Preferably, R¹ is C₄-C₃₀-alkyl, more preferably C₆-C₂₁-alkyl. It is even more preferred that the substituent (radical) R¹ is unsubstituted (in respect of all before-mentioned specific definitions). This means that the substituent R¹ is preferably straight-chain or branched.

In respect of the definition of the substituent R¹, it is also preferred that
i) R¹ is a mixture of at least two individual substituents, preferably R¹ is a mixture of at least two C₆-C₂₁-alkyl substituents, more preferably of at least two C₈-C₁₂-alkyl substituents, and/or
ii) R¹ is unsubstituted straight-chain or branched C₄-C₃₀-alkyl or C₄-C₃₀-alkenyl, preferably unsubstituted straight-chain or branched C₆-C₂₁-alkyl, more preferably unsubstituted straight-chain or branched C₈-C₁₂-alkyl.

It has to be noted that the before-mentioned option i) is exemplified below within working example 6, which is based on C₈-C₁₀ fatty acids. It also has to be noted that the above-mentioned option ii) in respect of unsubstituted straight-chain R¹ radicals is exemplified below, for example, within working example 1, whereas working example 3 is an example of an unsubstituted branched R¹ substituent. It has to be noted that the above-mentioned two options i) and ii) in respect of the definition of the substituent R¹ can, of course, be combined, for example, as a mixture of at least two unsubstituted straight-chain R¹ substituents, such as a substituent derived from unsubstituted straight-chain C₈-C₁₀ fatty acids. The same holds true in case at least one of the before-mentioned at least two R′ radicals is an unsubstituted branched R′ radical, which might also be the case in respect of a substituent derived from C₈-C₁₀ fatty acids.

The substituent R² is preferably C₃-C₁₂-alkylene, more preferably C₃-C₆-alkylene. It is even more preferred that the before-mentioned definitions of the substituent R² are unsubstituted, even more preferably straight-chain. By consequence, it is even more preferred that R² is straight-chain C₂-C₁₂-alkylene, preferably straight-chain C₃-C₆-alkylene.

In one embodiment of the present invention, the esteramine salts according to general formula (I) have an R² fragment, which is defined as —((CR¹⁰R¹¹)ₒ—CR⁴R⁵—CR⁶R⁷—O)ₘ—(CR⁸R⁹)ₙ—. The definitions of the substituents R⁴ to R¹¹, m, n and o are the same as defined above.

Within this embodiment, it is preferred that
R⁴, R⁵, R⁶, R⁷, R⁸, R⁹, R¹⁰ and R¹¹ are independently of each other selected from hydrogen or C₁-C₃-alkyl, more preferably hydrogen or methyl, most preferably hydrogen,
m is an integer from 1 to 10, preferably from 1 to 3,
n is an integer from 2 to 6, preferably 2 or 3, and
o is an integer from 0 to 5, preferably from 0 to 2.

Within this embodiment, it is even more preferred that the R² fragment is defined as follows:
R² is —(CH₂—CHR⁷—O)ₘ—CH₂—CHR⁹—, —(CHR¹¹)ₒ—CHR⁵—CHR⁷—O—(CH₂)₃— or —(CH₂—CH₂)ₚ—O—(CH₂—CH₂)ᵣ—,
R⁵, R⁷, R⁹ and R¹¹ are independently of each other selected from H or methyl, preferably R⁵, R⁷, R⁹ and R¹¹ are H,
m is an integer from 1 to 10, preferably m is 1,
n is an integer from 2 to 6, preferably n is 2,
o is an integer from 0 to 5, preferably o is 0 or 1,
p is an integer from 1 to 3, preferably p is 1,
r is an integer from 1 to 3, preferably r is 1.

R³ is preferably C₂-C₃₀-alkyl or at least monosubstituted aryl and the substituents are independently selected from C₁-C₃₀-alkyl under the proviso that R³ is not para toluenyl. R³ is more preferably C₆-C₁₈-alkyl or at least monosubstituted phenyl and the substituents are independently selected from C₁-C₃₀-alkyl under the proviso that R³ is not para toluenyl.

It is even more preferred that the substituent R³ is defined as follows:
i) R³ is monosubstituted phenyl and the substituent is in para position and selected from C₈-C₁₆-alkyl, and/or
ii) R³ is a mixture of at least two individual substituents, preferably of at least two isomers having a number of carbon atoms in the range of 8 to 20, more preferably of 16 to 18.

It has to be noted that the two before-mentioned options i) and ii) for the definition of the substituent R³ may be combined as exemplified below, for example, within working example 1.

It is therefore preferred that the substituent R³ is derived from dodecylbenzene sulfonic acid according to general formula (IVa), which is a mixture of isomers, wherein the respective alkyl fragments are in para position to the sulfonic acid group and m and n are independently of each other an integer from 0 to 10 under the proviso that the sum of m and n is an integer from 7 to 10.

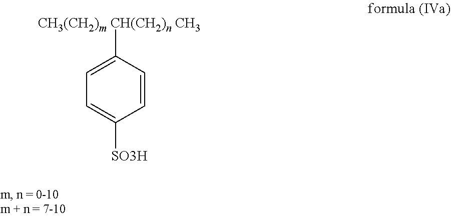

In one preferred embodiment of the present invention, the esteramine salt according to general formula (I) is defined as follows:
R¹ is C₄-C₃₀-alkyl,
R² is C₃-C₁₂-alkylene and
R³ is C₂-C₃₀-alkyl or at least monosubstituted aryl and the substituents are independently selected from C₁-C₃₀-alkyl under the proviso that R³ is not para toluenyl.

Within this embodiment, it is even more preferred that
R¹ is C₆-C₂₁-alkyl,
R² is C₃-C₆-alkylene and
R³ is C₆-C₁₈-alkyl or at least monosubstituted phenyl and the substituents are independently selected from C₁-C₃₀-alkyl under the proviso that R³ is not para toluenyl.

In another embodiment of the present invention, the esteramine salt according to the general formula (I) is defined as follows:
R¹ is C₄-C₃₀-alkyl,
R² is —(CH₂—CHR⁷—O)ₘ—CH₂—CHR⁹—, —(CHR¹¹)ₒ—CHR⁵—CHR⁷—O—(CH₂)₃— or —(CH₂—CH₂)ₚ—O—(CH₂—CH₂)ᵣ—,
R³ is C₂-C₃₀-alkyl or at least monosubstituted aryl and the substituents are independently selected from C₁-C₃₀-alkyl under the proviso that R³ is not para toluenyl, and
R⁵, R⁷, R⁹ and R¹¹ are independently of each other selected from H or methyl, preferably R⁵, R⁷, R⁹ and R¹¹ are H,
m is an integer from 1 to 10, preferably m is 1,
n is an integer from 2 to 6, preferably n is 2,
o is an integer from 0 to 5, preferably o is 0 or 1,
p is an integer from 1 to 3, preferably p is 1,
r is an integer from 1 to 3, preferably r is 1.

Another subject of the present invention is a process for preparing the esteramine salt as described above. Within this process for preparing an esteramine salt, a monocarboxylic acid or an ester thereof is reacted with an aminoalcohol and a sulfonic acid, and the molar ratio of sulfonic acid versus aminoalcohol is ≥1:1 [mol]/[mol]. The before-mentioned compounds as such (educts) are known to a person skilled in the art.

It has to be noted that the educts to be employed within the inventive process (i) monocarboxylic acid or an ester thereof, ii) aminoalcohol and iii) sulfonic acid) can be added to each other and/or mixed with each other in any amount or any ratio or any sequence/order as known to a person skilled in the art. For example, all educts can be mixed with each other in a first step, prior to initiating the process for preparing the esteramine salt according to the present invention. During this mixing step, the temperature should preferably be kept in a range of 20 to 90° C. After completion of the adding/mixing of all educts, the temperature is usually raised further, preferably to a range of 120 to 150° C. However, it is also possible that some or all of the educts of the inventive process are added step- and/or batchwise.

In case an ester of a monocarboxylic acid is employed within the inventive process, it is also possible that the respective ester is based on a bi- or higher functional alcohol, preferably on the trifunctional alcohol glycerine. By consequence, it is also possible that the respective alcohol fragment of said ester is connected with two or more individual monocarboxylic acid fragments. However, it is preferred that the respective ester, in particular the respective triglyceride is based on glycerine, and the respective monocarboxylic acid fragments are identical for each of the three ester groups contained within said compound.

Within this process, it is preferred that
i) the molar ratio of sulfonic acid versus aminoalcohol is from 1:1 to 2:1 [mol]/[mol], preferably from 1:1 to 1.5:1 [mol]/[mol], more preferably from 1.05:1 to 1.2:1 [mol]/[mol], and/or
ii) the molar ratio of carbonic acid or an ester thereof versus aminoalcohol is from 5:1 to 1:1 [mol]/[mol], preferably from 3:1 to 1.5:1 [mol]/[mol], more preferably from 1.5:1 to 1:1 [mol]/[mol].

The process according to the present invention is preferably carried out, comprising the steps a) to d) as follows:
a) the monocarboxylic acid or an ester thereof is mixed with an aminoalcohol, preferably at a temperature between 20 to 45° C.,
b) the sulfonic acid is added afterwards, preferably at a rate that the temperature of the reaction mixture does not exceed 90° C., more preferably the temperature of the reaction mixture does not exceed 80° C.,
c) after completion of the addition of sulfonic acid, the reaction mixture is heated further, preferably to a temperature in the range of 120 to 150° C. and/or for a time of 4 to 24 hours and
d) formed water or formed alcohol is optionally distilled out of the reaction mixture, preferably under vacuum.

In case the ester employed within step a) as described above is a triglyceride, it is preferred that step d) is not carried out since the released glycerine (formed alcohol from the employed triglyceride) preferably remains within the reaction mixture.

The monocarboxylic acid or an ester thereof to be employed within the inventive process are preferably defined as follows:
the monocarboxylic acid has the general formula (IIa)

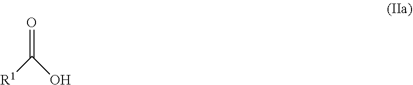

or an ester thereof has the general formula (IIb)

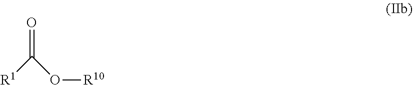

wherein
R¹ is C₄-C₃₀-alkyl or C₄-C₃₀-alkenyl, and
R¹⁰ is C₁-C₃₀-alkyl, preferably C₁-C₄-alkyl, or R¹⁰ is a fragment of a triglyceride.

An example of a monocarboxylic acid is decanoic acid or 3,3,5-trimethylhexane acid and C₈-C₁₀-fatty acid methyl ester is an example for an ester (methylester) of a monocarboxylic acid (C₈-C₁₀-fatty acid).

The aminoalcohol to be employed within the inventive process is preferably defined as follows:
the aminoalcohol has the general formula (III)

HO—R²—NH₂ (III)
wherein
R² is C₃-C₁₂-alkylene or —((CR¹⁰R¹¹)ₒ—CR⁴R⁵—CR⁶R⁷—O)ₘ—(CR⁸R⁹)ₙ—,
R⁴, R⁵, R⁶, R⁷, R⁸, R⁹, R¹⁰ and R¹¹ are independently of each other selected from hydrogen or C₁-C₁₀-alkyl,
m is an integer from 1 to 100,
n is an integer from 2 to 12, and
o is an integer from 0 to 10.

In one embodiment according to the inventive process, the aminoalcohol according to formula (III) is selected from an aminoalcohol, wherein R² is C₃-C₁₂-alkylene. 3-amino-1-propanol or 5-amino-1-pentanol are examples of such an aminoalcohol.

In another embodiment according to the inventive process, the aminoalcohol according to formula (III) is selected from an aminoalcohol, wherein R² is —((CR¹⁰R¹¹)ₒ—CR⁴R⁵—CR⁶R⁷—O)ₘ—(CR⁸R⁹)ₙ— and R⁴, R⁵, R⁶, R⁷, R⁸, R⁹, R¹⁰ and R¹¹ are independently of each other selected from hydrogen or C₁-C₁₀-alkyl,

m is an integer from 1 to 100,

n is an integer from 2 to 12, and

o is an integer from 0 to 10.

Such aminoalcohols according to formula (III), wherein R³ is —((CR¹⁰R¹¹)ₒ—CR⁴R⁵—CR⁶R⁷—O)ₘ—(CR⁸R⁹)ₙ—, are commercially available and may, for example, be obtained from the reaction of ammonia with C₃-C₁₆-alkylene oxide (as described in M. Frauenkron et al., ULLMANN'S Encyclopedia of Industrial Chemistry: “Ethanolamines and Propanolamines” 2001), or by reaction from ethylene glycols with acrylonitrile, followed by hydrogenation (e.g. described in DE2136884). Other routes to aminoalcohols according to formula (III) involve partial amination of polyglycol ethers with ammonia. 2-(2-aminoethoxy)ethanol is an example of an aminoalcohol falling under the definition of R² according to this embodiment.

The sulfonic acid to be employed within the inventive process is preferably defined as follows:
the sulfonic acid has the general formula (IV)

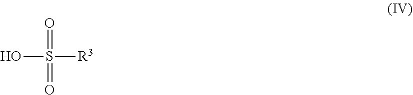

wherein
R³ is C₂-C₃₀-alkyl, C₂-C₃₀-alkenyl or unsubstituted or at least monosubstituted aryl and the substituents are independently selected from C₁-C₃₀-alkyl under the proviso that R³ is not para toluenyl.

A preferred example of a sulfonic acid is depicted in general formula (IVa)

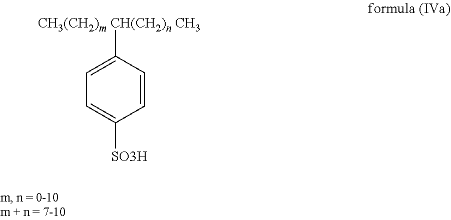

which is a mixture of isomers, wherein the respective alkyl fragments are in para position to the sulfonic acid group and m and n are independently of each other an integer from 0 to 10 under the proviso that the sum of m and n is an integer from 7 to 10.

Another example of a sulfonic acid is 2,4-dimethylbenzene sulfonic acid.

For the sake of completeness, it is indicated that further preferred, more preferred etc. definitions for the compounds as such (educts) to be employed within the inventive process are those which are in accordance with the respective preferred, more preferred etc. definitions for the esteramine salt according to general formula (I) as defined above.

It is also possible that the inventive process is carried out by additionally employing a solvent. Any solvent known to a skilled person may be employed, for example, water, xylene, toluene etc.

However, it is preferred that no additional solvent is employed within the inventive process.

The inventive process can be carried out within any apparatus known to a skilled person. The inventive process may also be carried out under an inert gas atmosphere, such as nitrogen or argon. Further aspects for carrying out the inventive process are exemplified below within the experimental part.

The effects for laundry as described and exemplified herein may be extrapolated to personal care applications.

The esteramine salts according to the present invention can be used and may be included in applications in personal care, as curing agent for epoxy resins, as reactant in the production of polymers, in polyurethanes, polyureas, and as thermoplastic polyamide adhesives. They can also be used in shampoo and body wash formulations. The esteramine salts may be included in personal care composition. By consequence, the before-mentioned use of the inventive esteramine salts as well as personal care compositions containing the inventive esteramine salts are further subjects of the present invention.

The following examples shall further illustrate the present invention without restricting the scope of this invention.

### EXAMPLE 1: DECANOIC ACID, ESTER WITH 3-AMINO-1-PROPANOL AS DODECYLBENZENE SULFONIC ACID SALT

In a 4-neck vessel with thermometer, reflux condenser, nitrogen inlet, dropping funnel, and stirrer, 11.3 g 3-amino-1-propanol and 25.8 g decanoic acid are placed at room temperature to 42° C. To the mixture 51.5 g dodecylbenzene sulfonic acid (mixture of isomers wherein each isomer is based on a monosubstituted benzene sulfonic acid with the substituent in para position as shown in FIG. 4a) is added within 30 minutes. The temperature is allowed to rise to 80° C. during the addition. The reaction mixture is heated to 130° C. and is stirred for 4 hours at 130° C. Vacuum is applied (5 mbar) and the mixture is stirred for 16 hours at 130° C. 83.0 g of a brown viscous oil is obtained. ¹H-NMR in MeOD indicates 89% conversion to decanoic acid, ester with 3-amino-1-propanol as dodecylbenzene sulfonic acid salt.

### EXAMPLE 2: DECANOIC ACID, ESTER WITH 3-AMINO-1-PROPANOL AS M-XYLENE SULFONIC ACID SALT

In a 4-neck vessel with thermometer, reflux condenser, nitrogen inlet, and stirrer, 18.77 g 3-amino-1-propanol and 43.07 g decanoic acid are placed at room temperature and heated to 55° C. To the mixture 46.66 g m-xylene sulfonic acid (2,4-dimethylbenzene sulfonic acid) is added in portions within 30 minutes. The temperature is allowed to rise to 70° C. during the addition. The reaction mixture is heated to 130° C. and is stirred for 4 hours at 130° C. Vacuum is applied (5 mbar) and the mixture is stirred for 30 hours at 130° C. 98.0 g of a brown wax is obtained. ¹H-NMR in MeOD indicates 81% conversion to decanoic acid, ester with 3-amino-1-propanol as xylene sulfonic acid salt.

### EXAMPLE 3: 3,5,5-TRIMETHYLHEXANE ACID (ISONONANOIC ACID), ESTER WITH 3-AMINO-1-PROPANOL AS DODECYLBENZENE SULFONIC ACID SALT ACID SALT

In a 4-neck vessel with thermometer, distillation equipment, nitrogen inlet, dropping funnel, and stirrer, 15.02 g 3-amino-1-propanol and 31.65 g 3,5,5-trimethylhexane acid are placed at room temperature to 72° C. To the mixture 66.61 g dodecylbenzene sulfonic acid (mixture of isomers as described in example 1) is added within 1 hour. The temperature is allowed to rise to 65° C. during the addition. The reaction mixture is heated to 130° C. and is stirred for 4 hours at 130° C. The formed water is destilled off. Vacuum is applied (5 mbar) and the mixture is stirred for 22 hours at 138° C. 105.0 g of a brown viscous oil is obtained. ¹H-NMR in MeOD indicates 98% conversion to 3,5,5-trimethylhexane acid, ester with 3-amino-1-propanol as dodecylbenzene sulfonic acid salt.

### EXAMPLE 4: DECANOIC ACID, ESTER WITH 2-(2-AMINOETHOXY)ETHANOL AS DODECYLBENZENE SULFONIC ACID SALT

In a 4-neck vessel with thermometer, reflux condenser, nitrogen inlet, dropping funnel, and stirrer, 26.3 g 2-(2-aminoethoxy)ethanol and 43.1 g decanoic acid are placed at room temperature. To the mixture 83.3 g dodecylbenzene sulfonic acid (mixture of isomers as described in example 1) is added within 15 minutes. The temperature is allowed to rise to 60° C. during the addition. The reaction mixture is heated to 130° C. and is stirred for 4 hours at 130° C. Vacuum is applied (5 mbar) and the mixture is stirred for 22 hours at 130° C. 140.0 g of a brown viscous oil is obtained. ¹H-NMR in MeOD indicates 95% conversion to decanoic acid, ester with 2-(2-aminoethoxy)ethanol as dodecylbenzene sulfonic acid salt.

### EXAMPLE 5: 3,5,5-TRIMETHYLHEXANE ACID (ISONONANOIC ACID), ESTER WITH 2-(2-AMINOETHOXY)ETHANOL AS DODECYLBENZENE SULFONIC ACID SALT

In a 4-neck vessel with thermometer, reflux condenser, nitrogen inlet, dropping funnel, and stirrer, 26.3 g 2-(2-aminoethoxy)ethanol and 36.6 g 3,5,5-trimethylhexane acid are placed at room temperature. To the mixture 83.3 g dodecylbenzene sulfonic acid (mixture of isomers as described in example 1) is added within 15 minutes. The temperature is allowed to rise to 60° C. during the addition. The reaction mixture is heated to 130° C. and is stirred for 4 hours at 130° C. Vacuum is applied (350 mbar) and the mixture is stirred for 22 hours at 130° C. 142.0 g of a brown viscous oil is obtained. ¹H-NMR in MeOD indicates 90% conversion to 3,5,5-trimethylhexane acid, ester with 2-(2-aminoethoxy)ethanol as dodecylbenzene sulfonic acid salt.

### EXAMPLE 6: C8-10 FATTY ACIDS, ESTER WITH 3-AMINO-1-PROPANOL AS DODECYLBENZENE SULFONIC ACID SALT, SYNTHESIZED FROM C8-10 FATTY ACID METHYL ESTER

In a 4-neck vessel with thermometer, distillation equipment, nitrogen inlet, dropping funnel, and stirrer, 3.8 g 3-amino-1-propanol and 26.6 g C8-10 fatty acid methyl ester (Aqnique ME610G) are placed at room temperature to 135° C. To the mixture 16.7 g dodecylbenzene sulfonic acid (mixture of isomers as described in example 1) is added within 30 minutes. The reaction mixture is stirred for 6 hours at 135° C., while the formed methanol is distilled off. Vacuum is applied (200 mbar) and the mixture is stirred for additional 5 hours at 135° C. and 200 mbar. Vacuum is lowered to 5 mbar and excess C8-10 fatty acid methyl ester is removed by stirring for 1.5 hours at 130° C. and 5 mbar. 27.0 g of a brown viscous oil is obtained. ¹H-NMR in MeOD indicates 94% conversion to C8-10 fatty acids, ester with 3-amino-1-propanol as dodecylbenzene sulfonic acid salt.

### EXAMPLE 7: C8-10 FATTY ACIDS, ESTER WITH 5-AMINO-1-PENTANOL AS DODECYLBENZENE SULFONIC ACID SALT, SYNTHESIZED FROM C8-10 FATTY ACID METHYL ESTER

In a 4-neck vessel with thermometer, distillation equipment, nitrogen inlet, dropping funnel, and stirrer, 5.4 g 5-amino-1-pentanol and 26.6 g C8-10 fatty acid methyl ester (Aqnique ME610G) are placed at room temperature and are heated to 100° C. To the mixture 16.7 g dodecylbenzene sulfonic acid (mixture of isomers as described in example 1) is added within 10 minutes. The reaction mixture is stirred for 6 hours at 135° C., while the formed methanol is distilled off. Vacuum is applied (200 mbar) and the mixture is stirred for additional 6 hours at 135° C. and 200 mbar. Vacuum is lowered to 5 mbar and excess C8-10 fatty acid methyl ester is removed by stirring for 2 hours at 130° C. and 9 mbar. 28.0 g of a brown viscous oil is obtained. ¹H-NMR in MeOD indicates 83% conversion to C8-10 fatty acids, ester with 5-amino-1-pentanol as dodecylbenzene sulfonic acid salt.

### EXAMPLE 8: OCTANOIC ACID, ESTER WITH 3-AMINO-1-PROPANOL AS DODECYLBENZENE SULFONIC ACID SALT, SYNTHESIZED FROM GLYCERYL TRIOCTANOATE

In a 4-neck vessel with thermometer, distillation equipment, nitrogen inlet, dropping funnel, and stirrer, 11.3 g 3-amino-1-propanol and 23.5 g glyceryltrioctanoate are placed at room temperature. To the mixture 50.0 g dodecylbenzene sulfonic acid (mixture of isomers as described in example 1) is added within 10 minutes. The reaction mixture is stirred for 12 hours at 135° C. 80.0 g of a brown viscous oil is obtained. ¹H-NMR in MeOD indicates 63% conversion to octanoic acid, ester with 3-amino-1-propanol as dodecylbenzene sulfonic acid salt.

### COMPARATIVE EXAMPLE 1: 3,5,5-TRIMETHYLHEXANE ACID (ISONONANOIC ACID), ESTER WITH 3-AMINO-1-PROPANOL AS METHANE SULFONIC ACID SALT ACID SALT

In a 4-neck vessel with thermometer, distillation equipment, nitrogen inlet, dropping funnel, and stirrer, 22.5 g 3-amino-1-propanol are placed at room temperature. 47.5 g 3,5,5-trimethylhexane acid is added within 25 min. To the mixture 29.4 g methane sulfonic acid is added within 20 minutes. The temperature is allowed to rise to 60° C. during the addition. The reaction mixture is heated to 130° C. and is stirred for 4 hours at 130° C. The formed water is distilled off. Vacuum is applied (5 mbar) and the mixture is stirred for 22 hours at 135° C. 89.0 g of a brown solid is obtained. ¹H-NMR in MeOD indicates 91% conversion to 3,5,5-trimethylhexane acid, ester with 3-amino-1-propanol as methane sulfonic acid salt.

### Use as Additives in Detergents

Technical stain swatches of blue knitted cotton containing Bacon Grease were purchased from Warwick Equest Ltd. The stains were washed for 30 min in a launder-o-meter (manufactured by SDL Atlas) at room temperature using per canister 500 mL of washing solution, 20 metal balls and ballast fabrics. The washing solution contained 5000 ppm (2.5 g in 500 mL canister) of detergent composition DC1 (table 1). Water hardness was 2.5 mM (Ca²⁺:Mg²⁺ was 4:1). 75 ppm of additives (as shown in table 2) were added to the washing solution of each canister separately and in the amount as detailed below. In the additive content is considered content of pure active in the salt.

Amount of additive is defined as follows:

$$A = 0.075 \times weight of canister [ kg ] \times \frac{100}{content of active in salt [ \% ]}$$

After addition the pH value was re-adjusted to the pH value of washing solution without additive.

Standard colorimetric measurement was used to obtain L*, a* and b* values for each stain before and after the washing. From L*, a* and b* values the stain level were calculated as color difference ΔE (calculated according to DIN EN ISO 11664-4) between stain and untreated fabric.

Stain removal from the swatches was calculated as follows:

$$Stain Removal Index ( SRI ) = \frac{Δ {E}_{initial} - Δ {E}_{washed}}{Δ {E}_{initial}} \times 100$$

$$Δ {E}_{initial} = Stain level before washing \\ Δ {E}_{washed} = Stain level after washing$$

Stain level corresponds to the amount of grease on the fabric. The stain level of the fabric before the washing (ΔEᵢₙᵢₜᵢₐₗ) is high, in the washing process stains are removed and the stain level after washing is smaller (ΔE_{washed}). The better the stains have been removed the lower the value for ΔE_{washed} will be and the higher the difference will be to ΔEᵢₙᵢₜᵢₐₗ. Therefore, the value of stain removal index increases with better washing performance as shown in table 2 below.

TABLE 1
Detergent composition DC1

| Ingredients of liquid detergent | percentage |
|---|---|
| composition DC1 | by weight |
| n-C₁₀-C₁₃-alkylbenzene sulfonic acid | 5.3 |
| coconut C₁₂-C₁₈ fatty acid | 2.4 |
| sodium laureth sulfate + 2 EO | 7.7 |
| potassium hydroxide | 2.2 |
| C₁₃C₁₅- oxo alcohol + 7 EO | 5.4 |
| 1,2 propylene glycol | 6 |
| ethanol | 2 |
| water | To Balance |
| pH of detergent composition DC1 = 8.0 | |

TABLE 2
Results of strain removal employing detergent composition
DC1 and additives

| | | | SRI, |
|---|---|---|---|
| | | | Bacon |
| | additives | | Grease |
| # | to DC1 | name and amount of additive | Cleaning |
| 1 | none | | 28.4 |
| 2 | Example 1 | 3-Amino-1-propanol, ester with | 44.2 |
| | | decanoic acid, 4-dodecylbenzene | |
| | | sulfonic acid (mixture of | |
| | | isomers) salt, 0.101 g per | |
| | | wash | |
| 3 | Example 3 | 3-Amino-1-propanol, ester with | 43.5 |
| | | 3,5,5-trimethylhexanoic | |
| | | acid, 4-dodecylbenzene | |
| | | sulfonic acid (mixture of isomers) | |
| | | salt, 0.095 g per wash | |
| 4 | Comparative | 3-Amino-1-propanol, ester with | 38.4 |
| | Example 1 | 3,5,5-trimethylhexanoic acid, | |
| | | methanesulfonic acid salt, | |
| | | 0.059 g per wash | |

As can be seen from table 2, especially form the comparison of experiments #3 and 4, strains can be removed more efficiently by employing a detergent composition DC1 containing a compound according to the present invention (example 3) compared to a composition containing comparative example 1 instead.

1. Esteramine salt according to general formula (I) 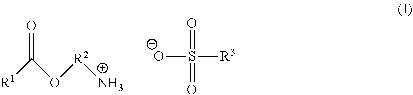
wherein:
R¹ is C₄-C₃₀-alkyl or C₄-C₃₀-alkenyl,
R² is C₃-C₁₂-alkylene or —((CR¹⁰R¹¹)ₒ—CR⁴R⁵—CR⁶R⁷—O)ₘ—(CR⁸R⁹)ₙ—,
R³ is C₂-C₃₀-alkyl, C₂-C₃₀-alkenyl or unsubstituted or at least monosubstituted aryl and the substituents are independently selected from C₁-C₃₀-alkyl under the proviso that R³ is not para toluenyl,
R⁴, R⁵, R⁶, R⁷, R⁸, R⁹, R¹⁰ and R¹¹ are independently of each other selected from hydrogen or C₁-C₁₀-alkyl,
m is an integer from 1 to 100,
n is an integer from 2 to 12, and
o is an integer from 0 to 10.

2. The esteramine salt according to claim 1, wherein
R¹ is C₄-C₃₀-alkyl,
R² is C₃-C₁₂-alkylene and
R³ is C₂-C₃₀-alkyl or at least monosubstituted aryl and the substituents are independently selected from C₁-C₃₀-alkyl under the proviso that R³ is not para toluenyl.

3. The esteramine salt according to claim 1, wherein
R¹ is C₆-C₂₁-alkyl,
R² is C₃-C₆-alkylene and
R³ is C₆-C₁₈-alkyl or at least monosubstituted phenyl and the substituents are independently selected from C₁-C₃₀-alkyl under the proviso that R³ is not para toluenyl.

4. The esteramine salt according to claim 1, wherein
i) R¹ is a mixture of at least two individual substituents and/or
ii) R¹ is unsubstituted straight-chain or branched C₄-C₃₀-alkyl or C₄-C₃₀-alkenyl.

5. The esteramine salt according to claim 1, wherein
i) R³ is monosubstituted phenyl and the substituent is in para position and selected from C₈-C₁₆-alkyl, and/or
ii) R³ is a mixture of at least two individual substituents.

6. The esteramine salt according to claim 1, wherein
i) R² is straight-chain C₂-C₁₂-alkylene, or
ii) R² is —(CH₂—CHR⁷—O)ₘ—CH₂—CHR⁹—, —(CHR¹¹)ₒ—CHR⁵—CHR⁷—O—(CH₂)₃— or —(CH₂—CH₂)ₚ—O—(CH₂—CH₂)ᵣ—,
R⁵, R⁷, R⁹ and R¹¹ are independently of each other selected from H or methyl,
m is an integer from 1 to 10,
n is an integer from 2 to 6,
o is an integer from 0 to 5,
p is an integer from 1 to 3,
is an integer from 1 to 3.

7. A process for preparing an esteramine salt according to claim 1,
wherein a monocarboxylic acid or an ester thereof is reacted with an aminoalcohol and a sulfonic acid, and the molar ratio of sulfonic acid versus aminoalcohol is ≥1:1 [mol]/[mol].

8. The process according to claim 7, wherein
i) the molar ratio of sulfonic acid versus aminoalcohol is from 1:1 to 2:1 [mol]/[mol], and/or
ii) the molar ratio of carbonic acid or an ester thereof versus aminoalcohol is from 5:1 to 1:1 [mol]/[mol].

9. The process according to claim 7, comprising the steps a) to d) as follows:
a) the monocarboxylic acid or an ester thereof is mixed with an aminoalcohol,
b) the sulfonic acid is added afterwards,
c) after completion of the addition of sulfonic acid, the reaction mixture is heated further, and
d) formed water or formed alcohol is optionally distilled out of the reaction mixture.

10. The process according to claim 7, wherein
the monocarboxylic acid has the general formula (IIa) 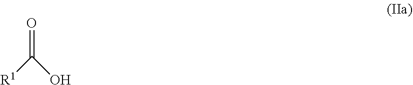
or an ester thereof has the general formula (IIb) 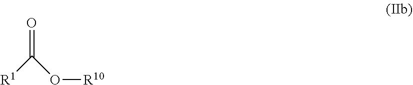
wherein
R¹ is C₄-C₃₀-alkyl or C₄-C₃₀-alkenyl, and
R¹⁰ is C₁-C₃₀-alkyl, or R¹⁰ is a fragment of a triglyceride.

11. The process according to claim 7, wherein
the aminoalcohol has the general formula (III)
HO—R²—NH₂ (III)
wherein
R² is C₃-C₁₂-alkylene or —((CR¹⁰R¹¹)ₒ—CR⁴R⁵—CR⁶R⁷—O)ₘ—(CR⁸R⁹)ₙ—,
R⁴, R⁵, R⁶, R⁷, R⁸, R⁹, R¹⁰ and R¹¹ are independently of each other selected from hydrogen or C₁-C₁₀-alkyl,
m is an integer from 1 to 100,
n is an integer from 2 to 12, and
o is an integer from 0 to 10.

12. The process according to claim 11, wherein
i) the aminoalcohol according to formula (III) is selected from an aminoalcohol, wherein R² is C₃-C₁₂-alkylene, or
ii) the aminoalcohol according to formula (III) is selected from an aminoalcohol, wherein R² is —((CR¹⁰R¹¹)ₒ—CR⁴R⁵—CR⁶R⁷—O)ₘ—(CR⁸R⁹)ₙ— and R⁴, R⁵, R⁶, R⁷, R⁸, R⁹, R¹⁰ and R¹¹ are independently of each other selected from hydrogen or C₁-C₁₀-alkyl,
m is an integer from 1 to 100,
n is an integer from 2 to 12, and
o is an integer from 0 to 10.

13. The process according to claim 7, wherein
the sulfonic acid has the general formula (IV) 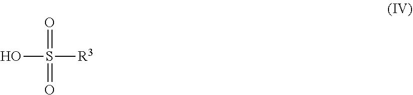
wherein
R³ is C₂-C₃₀-alkyl, C₂-C₃₀-alkenyl or unsubstituted or at least monosubstituted aryl and the substituents are independently selected from C₁-C₃₀-alkyl under the proviso that R³ is not para toluenyl.

14. Use of the esteramine salt according to claim 1 in personal care, as curing agent for epoxy resins, as reactant in the production of polymers, in polyurethanes, polyureas, and as thermoplastic polyamide adhesives.

15. Use of the esteramine salt according to claim 14 in shampoo and body wash formulations.

16. A personal care composition comprising the esteramine salt according to claim 1.
